(19) United States
(12) Patent Application Publication
(10) Pub. No.: US 2022/0361763 A1
(43) Pub. Date: Nov. 17, 2022

(54) TRACKING METHOD AND APPARATUS OF AN EXERCISE HEART RATE, DEVICE AND STORAGE MEDIUM

(71) Applicant: Guangdong COROS Sports Technology Joint Stock Company, Dongguan (CN)

(72) Inventors: Xin LIU, Dongguan (CN); Xuan RAO, Dongguan (CN); Haotian NIU, Dongguan (CN)

(21) Appl. No.: 17/737,713

(22) Filed: May 5, 2022

(30) Foreign Application Priority Data
May 12, 2021 (CN) 202110518148.4

(51) Int. Cl. A61B 5/024; A61B 5/00

(52) U.S. Cl. CPC A61B 5/02416; A61B 5/681; A61B 5/7285; A61B 5/746

(57) ABSTRACT

Provided are a tracking method and apparatus of an exercise heart rate, a device and a storage medium. The method includes: if the user heart rate currently detected by a photoelectric heart rate device does not meet a preset heart rate standard, invoking a pre-established target model equation reflecting the relationship between a user heart rate and a user pace; and inputting the current real-time pace of a user into the target model equation to obtain the current real-time heart rate of the user. In the technical solutions, when the user heart rate currently detected by the photoelectric heart rate device does not meet the preset heart rate standard, the current real-time pace of the user is directly input into the pre-established target model equation reflecting the relationship between the user heart rate and the user pace to obtain the current real-time heart rate of the user.

### CROSS-REFERENCE TO RELATED APPLICATION

This application claims priority to Chinese Patent Application No. 202110518148.4 filed May 12, 2021, the disclosure of which is incorporated herein by reference in its entirety.

### TECHNICAL FIELD

Embodiments of the present disclosure relate to the field of exercise data monitoring technology and, in particular, to a tracking method and apparatus of an exercise heart rate, a device and a storage medium.

### BACKGROUND

Since running is not restricted by factors such as sites and appliances, more and more users exercise through running. At this time, to prevent the users from excessive exercise during running, it is necessary to track exercise heart rates of the users during running in real time to ensure that the users can achieve the best exercise effect.

During running, the users usually use a wrist photoelectric heart rate device to detect the exercise heart rates of the users in real time. However, when the users are wearing the wrist photoelectric heart rate device, the device is affected by interference factors such as the sweat, environment and shaking during exercise, and detected exercise heart rates are very weak or disappear, resulting in errors in real-time tracking of the exercise heart rates. Thus, the detection of exercise heart rates during a subsequent running process is affected.

At present, in the period when the exercise heart rates tracked by the wrist photoelectric heart rate device in real time are very weak or disappear, an accelerometer is used to analyze the exercise state changes of the users before and after a moment to predict corresponding exercise heart rate changes. However, due to the large difference of heart rates of different users at the same exercise intensity, the adaptability is poor, and the difficulty of tracking the exercise heart rates is greatly increased.

### SUMMARY

Embodiments of the present disclosure provide a tracking method and apparatus of an exercise heart rate, a device and a storage medium.

In a first aspect, the embodiments of the present disclosure provide a tracking method of an exercise heart rate. The method includes steps described below.

If the user heart rate currently detected by a photoelectric heart rate device does not meet a preset heart rate standard, a pre-established target model equation reflecting the relationship between a user heart rate and a user pace is invoked.

The current real-time pace of a user is input into the target model equation to obtain the current real-time heart rate of the user.

In a second aspect, the embodiments of the present disclosure provide a tracking apparatus of an exercise heart rate. The apparatus includes a model equation invoking module and a heart rate tracking module.

The model equation invoking module is configured to, if the user heart rate currently detected by the photoelectric heart rate device does not meet the preset heart rate standard, invoke the target model equation reflecting the relationship between the user heart rate and the user pace.

The heart rate tracking module is configured to input the current real-time pace of the user into the target model equation to obtain the current real-time heart rate of the user.

In a third aspect, the embodiments of the present disclosure provide an electronic device. The electronic device includes one or more processors and a storage apparatus configured to store one or more programs.

When executed by the one or more processors, the one or more programs cause the one or more processors to perform the tracking method of an exercise heart rate according to any embodiment of the present disclosure.

In a fourth aspect, the embodiments of the present disclosure provide a computer-readable storage medium. The storage medium stores a computer program which, when executed by a processor, causes the processor to perform the tracking method of an exercise heart rate according to any embodiment of the present disclosure.

### BRIEF DESCRIPTION OF DRAWINGS

Other features, objects, and advantages of the present disclosure will become more apparent from a detailed description of non-restrictive embodiments with reference to the drawings described below.

FIG. 1 is a flowchart of a tracking method of an exercise heart rate according to embodiment one of the present disclosure.

FIG. 2 is a flowchart of a tracking method of an exercise heart rate according to embodiment two of the present disclosure.

FIG. 3 is a flowchart of a tracking method of an exercise heart rate according to embodiment three of the present disclosure.

FIG. 4 is a diagram illustrating the structure of a tracking apparatus of an exercise heart rate according to embodiment four of the present disclosure.

FIG. 5 is a diagram illustrating the structure of an electronic device according to embodiment five of the present disclosure.

### DETAILED DESCRIPTION

The present disclosure is further described hereinafter in detail in conjunction with drawings and embodiments. It is to be understood that the embodiments described herein are merely intended to explain the present disclosure and not to limit the present disclosure. Additionally, it is to be noted that for ease of description, only part, not all, of the structures related to the present disclosure are illustrated in the drawings.

### Embodiment One

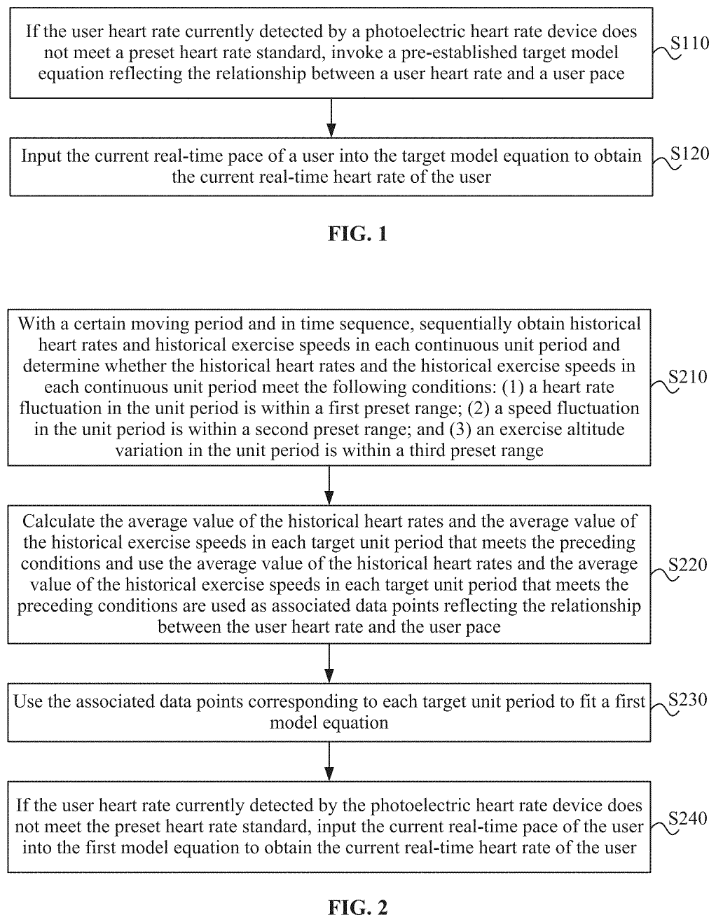

FIG. 1 is a flowchart of a tracking method of an exercise heart rate according to embodiment one of the present disclosure. This embodiment may be applied to the case of tracking the heart rate of any user during exercise. The tracking method of an exercise heart rate according to this embodiment may be executed by a tracking apparatus of an exercise heart rate according to this embodiment of the present disclosure. This apparatus may be performed by software and/or hardware and may be integrated in an electronic device performing this method.

In an embodiment, referring to FIG. 1, this method includes steps described below.

In S110, if the user heart rate currently detected by a photoelectric heart rate device does not meet a preset heart rate standard, a pre-established target model equation reflecting the relationship between a user heart rate and a user pace is invoked.

In an embodiment, since when a user is wearing the photoelectric heart rate device, the device is affected by interference factors such as the sweat, environment and shaking during user's exercise, and detected exercise heart rates are very weak or disappear, resulting in errors in real-time tracking of the exercise heart rate. Thus, the accurate detection of a real-time heart rate during a subsequent exercise process is affected. Therefore, in this embodiment, the parameter change related to the change of the user heart rate during the exercise needs to be analyzed to represent the change of the user heart rate with reference to the parameter change.

At this time, the parameter related to the heart rate change during the user's exercise is analyzed to know that the pace during the user's exercise may affect the change of the user heart rate. This pace may be expressed by the exercise speed of the user or an actual pace. Therefore, the change between the heart rate and the pace during the user's exercise is analyzed to determine the relationship between the user heart rate and the user pace. Thus, a target model equation reflecting the relationship between the user heart rate and the user pace may be established in advance. In this manner, a real-time pace of the user may be used to calculate a real-time heart rate of the user to achieve the real-time tracking of the user exercise heart rate.

In this embodiment, the target model equation is a first model equation fitted according to a historical heart rate and a historical exercise speed or a second model equation determined according to the relationship between a user heart rate reserve ratio and the user heart rate and the relationship between the user heart rate reserve ratio and the user pace. That is, during historical exercise, the user may detect the historical heart rate at each historical moment through the photoelectric heart rate device, and detect the historical exercise speed at the historical moment of each historical heart rate through a positioning device such as a global positioning system (GPS). Then a large number of historical heart rates and historical exercise speeds are used to fit the corresponding first model equation. In an embodiment, since the user heart rate reserve ratio can reflect the exercise intensity of the user in the current exercise, aerobic exercise and anaerobic exercise can be further determined. At this time, there is a corresponding calculation relationship between the user heart rate reserve ratio and the real-time heart rate of the user and the real-time pace of the user. Further, the relationship between the real-time heart rate of the user and the real-time pace of the user may be obtained through the user heart rate reserve ratio, and the corresponding second model equation is determined. At this time, through the first model equation and the second model equation, the real-time pace of the user may be used to calculate the corresponding real-time heart rate.

In this embodiment, during the user's exercise, the photoelectric heart rate device worn by the user is used to track the exercise heart rate of the user in real time. At this time, to ensure the tracking accuracy of the user exercise heart rate, this embodiment may set a preset heart rate standard. The user exercise heart rate tracked by the photoelectric heart rate device in real time needs to be able to meet the preset heart rate standard. The preset heart rate standard may be that the exercise heart rate is higher than a resting heart rate and changes within a certain range. For example, if the exercise heart rate currently detected by the photoelectric heart rate device is very weak or disappears directly, the exercise heart rate detected does not meet the preset heart rate standard.

Therefore, to achieve the real-time tracking of the user exercise heart rate, when the user heart rate currently detected by the photoelectric heart rate device does not meet the preset heart rate standard, the target model equation reflecting the relationship between the user heart rate and the user pace may be invoked. In this manner, the real-time pace of the user may be used to calculate the real-time heart rate of the user to ensure the accuracy of the user exercise heart rate.

In S120, the current real-time pace of the user is input into the target model equation to obtain the current real-time heart rate of the user.

In an embodiment, if the user heart rate currently detected by the photoelectric heart rate device does not meet the preset heart rate standard, the current real-time pace of the user may be detected by the positioning device such as the GPS. Then this real-time pace may be input into the established target model equation. The current real-time heart rate of the user may be calculated through this target model equation. At this time, for any user, the target model equation may be used to analyze the real-time heart rate during the user's exercise, to ensure the overall adaptability of the exercise heart rate tracking.

As an optional solution in this embodiment, when the current real-time pace of the user is detected by the positioning device such as the GPS, there may be an error in a user pace due to the positioning drift of the GPS. Therefore, to further improve the tracking accuracy of the exercise heart rate, that the current real-time pace of the user is input into the target model equation to obtain the current real-time heart rate of the user may specifically include: inputting the current real-time pace of the user into the target model equation to obtain a current initial heart rate of the user; and determining the current real-time heart rate of the user according to the difference between the initial heart rate and a real-time heart rate of the user at a previous moment.

That is, after the current real-time pace of the user is detected by the positioning device such as the GPS, the current real-time pace of the user may be input into the target model equation to obtain the current user heart rate. At this time, to avoid the error in the user pace due to the positioning drift of the GPS, the user heart rate output by the target model equation may be used as the current initial heart rate. Then, the current initial heart rate of the user is compared with the real-time heart rate of the user actually tracked at the previous moment to determine whether the difference between the two is too large. Therefore, whether there is the error in the user pace due to the positioning drift of the GPS is analyzed. If the difference between the two is too large, for example, the difference between the two exceeds a preset change index (for example, 3), the current heart rate change is directly limited to this preset change index. Then, based on the real-time heart rate at the previous moment, the preset change index is used to adjust accordingly to determine the current real-time heart rate of the user. Therefore, the error in the user pace due to the positioning drift of the GPS is filtered out. However, if the difference between the two is small, for example, the difference between the two does not exceed the preset change index, it is to be indicated that the GPS does not drift, this initial heart rate may be directly used as the current real-time heart rate of the user.

Additionally, if the deviation between the user heart rate currently tracked by the photoelectric heart rate device and its own actual heart rate is too large, the current real-time heart rate of the user may be measured by actively triggering electrocardiogram (ECG) technology. That is, to further ensure the tracking accuracy of the user exercise heart rate, in this embodiment, when the deviation between the user heart rate currently tracked by the photoelectric heart rate device and its own actual heart rate is too large, the user may be required to stop the exercise for about 30 s. An ECG device is used to measure the current real-time heart rate of the user to correct the user heart rate tracked by the photoelectric heart rate device. Thus, the photoelectric heart rate device can continue to use the real-time heart rate measured by the ECG device as a reference value to re-track a heart rate signal during the user's exercise. In this manner, an accurate exercise heart rate is obtained, and the accuracy of subsequent photoelectric heart rate tracking is improved.

In the technical solutions of this embodiment, when the user heart rate currently detected by the photoelectric heart rate device does not meet the preset heart rate standard, the current real-time pace of the user may be directly input into the pre-established target model equation reflecting the relationship between the user heart rate and the user pace to obtain the current real-time heart rate of the user. In this manner, the real-time tracking of the exercise heart rate is achieved, and the problem that the exercise heart rate tracked by the photoelectric heart rate device in real time is inaccurate is avoided. Thus, the overall adaptability of the exercise heart rate tracking is ensured, and the accuracy of the exercise heart rate tracking is improved.

### Embodiment Two

FIG. 2 is a flowchart of a tracking method of an exercise heart rate according to embodiment two of the present disclosure. This embodiment of the present disclosure is an optimization on the basis of the preceding embodiment. In an embodiment, the target model equation may be the first model equation fitted according to the historical heart rate and the historical exercise speed or the second model equation determined according to the relationship between the user heart rate reserve ratio and the user heart rate and the relationship between the user heart rate reserve ratio and the user pace. In this embodiment, the specific process of establishing the first model equation reflecting the relationship between the user heart rate and the user pace is mainly explained in detail.

In an embodiment, referring to FIG. 2, the method according to this embodiment may specifically include steps described below.

In S210, with a certain moving period and in time sequence, historical heart rates and historical exercise speeds in each continuous unit period are sequentially obtained and whether the historical heart rates and the historical exercise speeds in each continuous unit period meet the following conditions is determined. (1) A heart rate fluctuation in the unit period is within a first preset range. (2) A speed fluctuation in the unit period is within a second preset range. (3) An exercise altitude variation in the unit period is within a third preset range.

In this embodiment, when the first model equation fitted according to the historical heart rates and the historical exercise speeds is analyzed, to ensure the effectiveness of the obtained historical heart rates and historical exercise speeds, some data unrelated to exercise is filtered out to the greatest extent. Corresponding historical heart rate data and corresponding historical exercise speed data are collected in a moving average manner.

In an embodiment, at this time, the unit period specified by the moving average manner may be set, for example, a data point composed of the historical heart rates and the historical exercise speeds is collected every two minutes, and the moving period specified by the moving average manner may be set, for example, moving and collecting once per second. At this time, when the historical heart rates and the historical exercise speeds are collected, it is possible to continuously move in this moving period in time sequence. Each moving may be corresponding to one unit period. Then, each historical heart rate detected in the current unit period and the historical exercise speed detected at the same time as each historical heart rate are obtained. At this time, each historical heart rate and each historical exercise speed in each continuous unit period may be continuously obtained by continuously moving in a certain moving period.

In this embodiment, for example, the moving period is 1 second, and the unit period is 2 minutes. Moving obtainment refers to continuously obtaining multiple data such as historical heart rates and historical exercise speeds in each continuous unit period such as the 1st to 120th seconds, the 2nd to 121st seconds and the 3rd to 122nd seconds.

Then, to ensure the effectiveness of the historical heart rates and historical exercise speeds, it is necessary to determine whether the heart rate fluctuation of the historical heart rates in each unit period is within the first preset range (plus 5 to minus 5), whether the fluctuation of the historical exercise speeds in each unit period is within the second preset range (plus 0.5 m/s to minus 0.5 m/s), and whether the altitude variation in each unit period is within the third preset range (less than 3 m). Only when the data in each unit period meet the preceding three conditions at the same time, can effective historical heart rate data and effective historical exercise speed data be obtained.

In S220, the average value of the historical heart rates and the average value of the historical exercise speeds in each target unit period that meets the preceding conditions are calculated, and the average value of the historical heart rates and the average value of the historical exercise speeds in each target unit period that meets the preceding conditions are used as associated data points reflecting the relationship between the user heart rate and the user pace.

As an optional solution in this embodiment, a target unit period that meets the preceding three conditions at the same time may be selected from all unit periods. Then, for each target unit period, the average value of each historical heart rate and the average value of each historical exercise speed in this target unit period may be calculated respectively. Moreover, the average value of the historical heart rates and the average value of the historical exercise speeds may be jointly used as the associated data points reflecting the relationship between the user heart rate and the user pace in this target unit period. At this time, according to the preceding process, one associated data point reflecting the relationship between the user heart rate and the user pace may be determined in each target unit period to ensure the moving average relevance between the historical heart rate and the historical exercise speed.

In S230, the associated data points corresponding to each target unit period are used to fit the first model equation.

In this embodiment, the associated data points corresponding to the target unit periods within one month may be saved. The number of the associated data points is required to exceed a certain number (such as 30) to use the associated data points corresponding to each target unit period to fit the corresponding first model equation.

For example, the first model equation fitted in this embodiment may be: hr=k*v+b. hr denotes the real-time heart rate of the user. v denotes the real-time exercise speed of the user. k and b denote the fixed values of the equation fitted by using the associated data points corresponding to each target unit period.

In S240, if the user heart rate currently detected by the photoelectric heart rate device does not meet the preset heart rate standard, the current real-time pace of the user is input into the first model equation to obtain the current real-time heart rate of the user.

In the technical solutions of this embodiment, first, the associated data points composed of the average value of the historical heart rates and the average value of the historical exercise speeds in each target unit period are obtained in a moving average manner. In this manner, the first model equation reflecting the relationship between the user heart rate and the user pace is established to ensure the accuracy of the first model equation for the real-time heart rate tracking. Then, when the user heart rate currently detected by the photoelectric heart rate device does not meet the preset heart rate standard, the current real-time pace of the user may be directly input into the first model equation to obtain the current real-time heart rate of the user. In this manner, the real-time tracking of the exercise heart rate is achieved, and the problem that the exercise heart rate tracked by the photoelectric heart rate device in real time is inaccurate is avoided. Thus, the overall adaptability of the exercise heart rate tracking is ensured, and the accuracy of the exercise heart rate tracking is improved.

### Embodiment Three

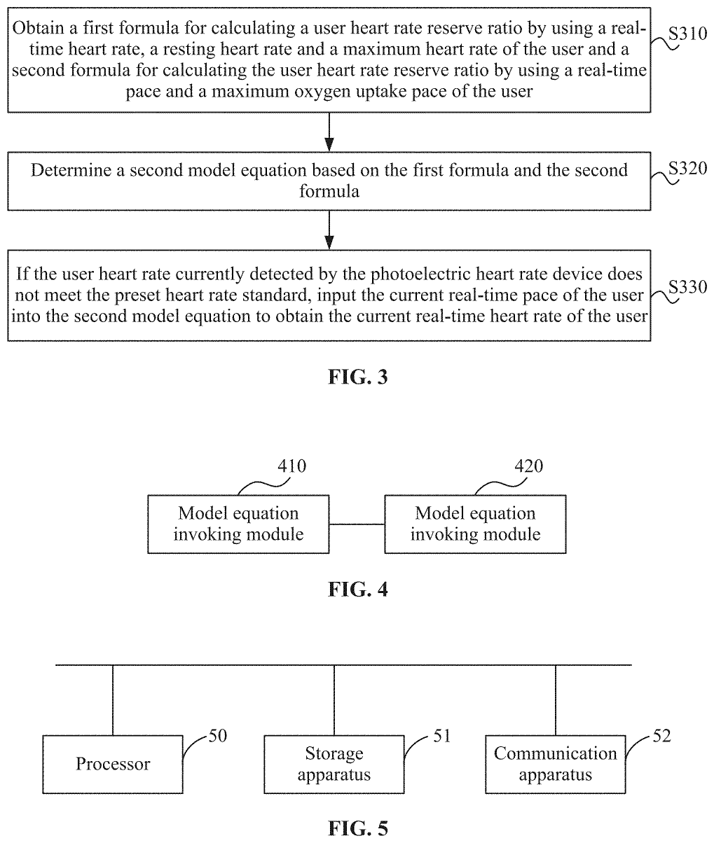

FIG. 3 is a flowchart of a tracking method of an exercise heart rate according to embodiment three of the present disclosure. This embodiment of the present disclosure is an optimization on the basis of the preceding embodiments. In an embodiment, the target model equation may be the first model equation fitted according to the historical heart rate and the historical exercise speed or the second model equation determined according to the relationship between the user heart rate reserve ratio and the user heart rate and the relationship between the user heart rate reserve ratio and the user pace. In this embodiment, the specific process of establishing the second model equation reflecting the relationship between the user heart rate and the user pace is mainly explained in detail.

In an embodiment, referring to FIG. 3, the method according to this embodiment may specifically include steps described below.

In S310, a first formula for calculating the user heart rate reserve ratio by using the real-time heart rate, the resting heart rate and the maximum heart rate of the user and a second formula for calculating the user heart rate reserve ratio by using the real-time pace and the maximum oxygen uptake pace of the user are obtained.

In an embodiment, the user heart rate reserve ratio may reflect the exercise intensity of the user. The first formula for calculating the user heart rate reserve ratio through the user heart rate may be:

$$ρ = \frac{{hr}_{i} - {hr}_{0}}{{hr}_{max} - {hr}_{0}} .$$

hr₁ denotes the real-time heart rate of the user during the exercise. hrₘₐₓ denotes the maximum heart rate of the user. hr₀ denotes the resting heart rate of the user in an awake and quiet state. At this time, the maximum heart rate and resting heart rate may be detected by a portable heart rate detection device. In a case where the user knows the value of his maximum heart rate and resting heart rate, the maximum heart rate and resting heart rate may also be set manually by the user. Additionally, the maximum heart rate may also be calculated through the user age, that is, hrₘₐₓ=208−0.7a. a denotes the user age.

At the same time, since the maximum oxygen uptake (VO2max) refers to the oxygen content that the human body can take in during the maximum intensity exercise, and when the exercise intensity approaches the maximum heart rate, the exercise intensity may also approaches the maximum oxygen uptake. Therefore, the maximum oxygen uptake pace can also represent the pace that the user can achieve at the maximum exercise intensity. At this time, the user heart rate reserve ratio can also reflect the exercise intensity of the user through the maximum oxygen uptake pace. In this embodiment, the second formula for calculating the user heart rate reserve ratio through the user pace may be:

$$ρ = \frac{{pace}_{i}}{{pace}_{max}} .$$

pace₁ denotes the real-time pace of the user during the exercise. paceₘₐₓ X denotes the maximum oxygen uptake pace of the user. At this time, the maximum oxygen uptake pace of the user is calculated according to the exercise condition during the historical exercise. In a case where the user knows his maximum oxygen uptake pace, the maximum oxygen uptake pace may also be set manually by the user.

In S320, the second model equation is determined based on the first formula and the second formula.

In an embodiment, after the first formula and the second formula are obtained, in combination with the user heart rate reserve ratio, the second model equation may be determined as:

$${hr}_{i} = \frac{{pace}_{i}}{{pace}_{max}} * ( {hr}_{max} - {hr}_{0} ) + {hr}_{0} .$$

Through this second model equation, the current real-time pace of the user may be used to calculate the current real-time heart rate of the user.

It is to be noted that in the second model equation, the maximum heart rate and the resting heart rate can be determined in advance, and when the maximum oxygen uptake pace is unknown in advance, a certain calculation needs to be performed.

In this embodiment, the calculation process of the maximum oxygen uptake pace may specifically include: determining a corresponding lactate threshold pace by using the standard pace of the user in a standard competition course; and determining the maximum oxygen uptake pace of the user based on the corresponding lactate threshold pace.

In an embodiment, the standard competition course may be divided into a half marathon course and a full marathon course. At this time, the actual exercise condition of the user in the standard competition course is analyzed to analyze the standard pace of the user in the standard competition course.

At this time, as shown in Table 1, a matching data table between a large number of standard paces, lactate threshold paces and maximum oxygen uptake paces may be collected in advance during the historical exercise of each user in the standard competition course.

TABLE 1
Matching data table between standard pace, lactate threshold
pace and maximum oxygen uptake pace in half marathon course

| | Lactate | Maximum Oxygen |
|---|---|---|
| Standard Pace | Threshold Pace | Uptake Pace |
| 2:45 | 2:44 | 2:44/120% |
| 3:25 | 3:24 | 3:24/120% |
| 4:05 | 4:02 | 4:02/120% |
| 4:47 | 4:40 | 4:40/118% |
| 5:30 | 5:16 | 5:16/117% |
| 6:13 | 6:03 | 6:03/115% |
| 6:52 | 6:27 | 6:27/114% |
| 7:25 | 7:15 | 7:15/113% |
| 8:03 | 7:40 | 7:40/112% |
| 8:55 | 8:20 | 8:20/111% |
| 9:29 | 9:10 | 9:10/110% |

In different competition courses, the corresponding preceding data may be obtained. At this time, according to the corresponding data in the preceding table in each standard competition course, a first association equation reflecting the relationship between the standard pace and the lactate threshold pace and the second association equation reflecting the relationship between the lactate threshold pace and the maximum oxygen uptake pace in the standard competition course may be fitted. Therefore, the standard pace of the user in the standard competition course is input into the first association equation to obtain the lactate threshold pace corresponding to this standard pace. Then, this lactate threshold rate is input into the second association equation to obtain the maximum oxygen uptake pace corresponding to this lactate threshold pace.

Further, the calculation process of the standard pace of the user in the standard competition course may specifically include: determining the target heart rate reserve ratio by using the maximum oxygen uptake of the user in the standard competition course; calculating the standard exercise heart rate of the user in the standard competition course based on the target heart rate reserve ratio and the maximum heart rate and the resting heart rate of the user; and calculating the standard pace of the user in the standard competition course by using the standard exercise heart rate and each pre-established piecewise model equation reflecting the relationship between the user heart rate and the user pace in the standard competition course.

In this embodiment, the physiological characteristics of the user such as the age, gender and weight, the maximum heart rate and resting heart rate of the user in the standard competition course, and the average exercise heart rate and the average exercise speed calculated by using the real-time exercise heart rate and real-time exercise speed during a period of exercise in the standard competition course may be analyzed to obtain the maximum oxygen uptake supported by the user in the standard competition course. In this embodiment, the calculation formula of the maximum oxygen uptake may be:

$${V}_{O , max} = A + {P}_{1} * S - {P}_{2} * G + {P}_{3} * V * \frac{{P}_{A}}{B} - C * \frac{{hr}_{avg} - {hr}_{0}}{{hr}_{max} - {hr}_{0}} - \frac{2 ( a - 26 )}{5} .$$

A denotes a constant ranging from 40 to 50. P₁ denotes a constant ranging from 7 to 8. S denotes a gender constant, 1 for males and 0 for females. P₂ denotes a constant ranging from 0.1 to 0.2. G is the user weight. P₃ denotes a constant ranging from 4 to 5. V denotes the average exercise speed. P₄ denotes a constant ranging from 3 to 4. B denotes a constant ranging from 1 to 2. C denotes a constant ranging from 15 to 20. hr_{avg} denotes the average exercise heart rate. hr₀ denotes the resting heart rate of the user in an awake and quiet state. hrₘₐₓ denotes the maximum heart rate of the user. a denotes the user age.

Further, according to the exercise in the competition course, a heart rate reserve ratio query table corresponding to the standard competition course can be made. A half marathon course is used as an example. As shown in Table 2 and Table 3, the user heart rate reserve ratio query table records heart rate reserve ratios corresponding to users having different physiological characteristics and maximum oxygen uptake relative to the half marathon course.

TABLE 2
Heart rate reserve ratio of male in half marathon course

| | | 78% (Any Percentage | |
|---|---|---|---|
| | | ranging from 75% to | |
| | 75% | 80%) | 80% |
| | Maximum | Maximum | Maximum |
| | Oxygen | Oxygen | Oxygen |
| | Uptake Pace | Uptake Pace | Uptake Pace |
| Age (Male) | Zone 1 | Zone 2 | Zone 3 |
| 29 and below | 45 and below | 46-59 | 60 and above |
| 30-34 | 43 and below | 44-57 | 58 and above |
| 35-39 | 41 and below | 42-55 | 56 and above |
| 40-44 | 39 and below | 40-53 | 54 and above |
| 45-49 | 37 and below | 38-51 | 52 and above |
| 50-54 | 35 and below | 36-49 | 50 and above |
| 55-59 | 33 and below | 34-47 | 48 and above |
| 60-64 | 31 and below | 32-45 | 46 and above |
| 65 and above | 29 and below | 30-43 | 43 and above |

TABLE 3
Heart rate reserve ratio of female in half marathon course

| | | 78% (Any Percentage | |
|---|---|---|---|
| | | ranging from 75% to | |
| | 75% | 80%) | 80% |
| | Maximum | Maximum | Maximum |
| | Oxygen | Oxygen | Oxygen |
| | Uptake Pace | Uptake Pace | Uptake Pace |
| Age (Female) | Zone 1 | Zone 2 | Zone 3 |
| 29 and below | 38 and below | 39-52 | 53 and above |
| 30-34 | 36 and below | 37-50 | 51 and above |
| 35-39 | 34 and below | 35-48 | 49 and above |
| 40-44 | 32 and below | 33-46 | 47 and above |
| 45-49 | 30 and below | 31-44 | 45 and above |
| 50-54 | 28 and below | 29-42 | 43 and above |
| 55-59 | 26 and below | 27-40 | 41 and above |
| 60-64 | 24 and below | 25-38 | 39 and above |
| 65 and above | 22 and below | 23-36 | 37 and above |

For different standard competition courses, the heart rate reserve ratio query table corresponding to the preceding half marathon course may be made. At this time, the heart rate reserve ratio query table in the standard competition course may be queried according to the physiological characteristic data and maximum oxygen uptake of the user to obtain the target heart rate reserve ratio of the user relative to the standard competition course.

Then, based on the calculation formula of the heart rate reserve ratio and the maximum heart rate and resting heart rate of the user, the standard exercise heart rate of the user in the standard competition course may be calculated.

At this time, to use the standard exercise heart rate to calculate the standard pace of the user in the standard competition course, it is necessary to select a large number of effective historical heart rates and historical paces to establish a model equation reflecting the relationship between the user heart rate and the user pace in the standard competition course. However, since the standard competition course is divided into different competition courses such as a half marathon course and a full marathon course, to calculate the standard pace in each standard competition course, in this embodiment, all kinds of data collected may be divided according to an exercise distance, and the collected data is divided into five pieces: 0-10 km, 10-20 km, 20-30 km, 30-40 km and more than 40 km. The corresponding piecewise model equation can be established in each piece, which is convenient for the combination of each piece to obtain different standard competition courses. Thus, standard paces of different standard competition courses are calculated.

For example, in this embodiment, data of the user such as the real-time heart rate, real-time pace, temperature, humidity, altitude, slope, distance, body temperature and exercise time during the historical exercise may be used for reference, and the heart rate data and the pace data in line with the model are selected. The specific requirements are described below.

(1) The single exercise duration of the user is greater than set duration (for example, 10 minutes).

(2) With a certain moving period and in time sequence, the historical heart rates and the historical exercise speeds in each continuous unit period are sequentially obtained and whether the historical heart rates and the historical exercise speeds in each continuous unit period meet the following conditions is determined. a. The heart rate fluctuation in the unit period is within a preset range (not exceeding 12 bpm). b. The speed fluctuation in the unit period is within another preset range (not exceeding 5 m/s). c. The heart rate reserve ratio in the unit period is within a preset interval (50% to 90%). At this time, the historical heart rates and the historical paces in each target unit period that meet the preceding conditions may be continuously obtained, and the average value of the historical heart rates and the average value of the historical paces in each target unit period are calculated, and the average value of the historical heart rates and the average value of the historical paces in each target unit period are used as the corresponding associated data points. For example, the unit period is 2 minutes, and the moving period is 1 second. The heart rates and the paces in every 2 minutes are continuously recorded. The average value of the heart rates and the average value of the paces in every 2 minutes are calculated, that is, one 2-minute unit period is counted every 1 second. Therefore, multiple data such as historical heart rates and historical exercise speeds in each continuous unit period such as the 1st to 120th seconds, the 2nd to 121st seconds and the 3rd to 122nd seconds is continuously obtained.

Then, according to each exercise distance, the associated data points composed of the historical heart rates and the historical paces in each target unit period are classified to form the corresponding data sets in the five pieces of 0-10 km, 10-20 km, 20-30 km, 30-40 km and more than 40 km. Therefore, the data set in each piece can be used to fit the piecewise model equation that is in this piece and reflects the relationship between the user heart rate and the user pace. For example, the piecewise model equation in each piece may be:

$${pace}_{i} = \frac{{hr}_{i} - B}{A} .$$

This equation is used to calculate the corresponding standard pace by using the standard exercise heart rate of the user in the standard competition course. Alternatively, to ensure the accuracy of standard pace, the piecewise model equation may also be:

$${pace}_{i} = \frac{{hr}_{i} + time * C + atmos * D + humi * E + hei * F + slope * G + temp * H - B}{A} .$$

time denotes the exercise time point. atmos denotes the atmospheric temperature. humi denotes the humidity. hei denotes the altitude. slope denotes the slope. temp denotes the body temperature. The accuracy of each piecewise model equation is ensured by adding influence parameters.

At this time, through the analysis of the preceding historical data, the piecewise model equations reflecting the relationship between the user heart rate and the user pace in the five pieces of 0-10 km, 10-20 km, 20-30 km, 30-40 km and more than 40 km may be established. At this time, a piecewise model equation related to the standard competition course may be selected from the five piecewise model equations. Then, the standard exercise heart rate of the user in the standard competition course may be input into each piecewise model equation related to the standard competition course to calculate the corresponding standard pace.

For example, the half marathon course is used as the standard competition course, and that the piecewise model equations in the piece of 0-10 km and the piece of 10-20 km are related to the half marathon course may be determined. At this time, the standard exercise heart rate of the user in the half marathon course is input into the piecewise model equations in the piece of 0-10 km and the piece of 10-20 km respectively. A first pace pace₁ the piece of 0-10 km and a second pace pace₂ the piece of 10-20 km may be obtained respectively, and a half marathon pace may be calculated as

$$\frac{{pace}_{1} * 10 + {pace}_{2} * 10}{20} .$$

With reference to the preceding steps, the standard pace of the user in the standard competition course may be calculated through each piecewise model equation and the standard exercise heart rate in the standard competition course. Therefore, the standard pace in the standard competition course is used to determine the corresponding lactate threshold pace. The maximum oxygen uptake pace of the user is determined based on this lactate threshold pace. Thus, this maximum oxygen uptake pace is substituted into the second model equation, and the current real-time pace of the user is used to calculate the current real-time heart rate of the user.

In S330, if the user heart rate currently detected by the photoelectric heart rate device does not meet the preset heart rate standard, the current real-time pace of the user is input into the second model equation to obtain the current real-time heart rate of the user.

In the technical solutions of this embodiment, first, based on the first formula for calculating the user heart rate reserve ratio by using the real-time heart rate, the resting heart rate and the maximum heart rate of the user and the second formula for calculating the user heart rate reserve ratio by using the real-time pace and the maximum oxygen uptake pace of the user, the second model equation reflecting the relationship between the user heart rate and the user pace is established. The accuracy of the second model equation for the real-time heart rate tracking is ensured. Then, when the user heart rate currently detected by the photoelectric heart rate device does not meet the preset heart rate standard, the current real-time pace of the user may be directly input into the second model equation to obtain the current real-time heart rate of the user. In this manner, the real-time tracking of the exercise heart rate is achieved, and the problem that the exercise heart rate tracked by the photoelectric heart rate device in real time is inaccurate is avoided. Thus, the overall adaptability of the exercise heart rate tracking is ensured, and the accuracy of the exercise heart rate tracking is improved.

### Embodiment Four

FIG. 4 is a diagram illustrating the structure of a tracking apparatus of an exercise heart rate according to embodiment four of the present disclosure. As shown in FIG. 4, the apparatus may include a model equation invoking module 410 and a heart rate tracking module 420.

The model equation invoking module 410 is configured to, if the user heart rate currently detected by the photoelectric heart rate device does not meet the preset heart rate standard, invoke the target model equation reflecting the relationship between the user heart rate and the user pace.

The heart rate tracking module 420 is configured to input the current real-time pace of the user into the target model equation to obtain the current real-time heart rate of the user.

In the technical solutions of this embodiment, when the user heart rate currently detected by the photoelectric heart rate device does not meet the preset heart rate standard, the current real-time pace of the user may be directly input into the pre-established target model equation reflecting the relationship between the user heart rate and the user pace to obtain the current real-time heart rate of the user. In this manner, the real-time tracking of the exercise heart rate is achieved, and the problem that the exercise heart rate tracked by the photoelectric heart rate device in real time is inaccurate is avoided. Thus, the overall adaptability of the exercise heart rate tracking is ensured, and the accuracy of the exercise heart rate tracking is improved.

Further, the preceding target model equation is the first model equation fitted according to the historical heart rate and the historical exercise speed or the second model equation determined according to the relationship between the user heart rate reserve ratio and the user heart rate and the relationship between the user heart rate reserve ratio and the user pace.

Further, if the preceding target model equation is the first model equation, the preceding target model equation may be established using the steps below.

With a certain moving period and in time sequence, the historical heart rates and the historical exercise speeds in each continuous unit period are sequentially obtained and whether the historical heart rates and the historical exercise speeds in each continuous unit period meet the following conditions is determined.

(1) The heart rate fluctuation in each continuous unit period is within the first preset range.

(2) The speed fluctuation in each continuous unit period is within the second preset range.

(3) The altitude variation in each continuous unit period is within the third preset range.

The average value of the historical heart rates and the average value of the historical exercise speeds in each target unit period that meets the preceding conditions are calculated, and the average value of the historical heart rates and the average value of the historical exercise speeds in each target unit period that meets the preceding conditions are used as the associated data points reflecting the relationship between the user heart rate and the user pace.

The associated data points corresponding to each target unit period is used to fit the first model equation.

Further, if the preceding target model equation is the second model equation, the preceding target model equation may be established using the steps below.

The first formula for calculating the user heart rate reserve ratio by using the real-time heart rate, the resting heart rate and the maximum heart rate of the user and the second formula for calculating the user heart rate reserve ratio by using the real-time pace and the maximum oxygen uptake pace of the user are obtained.

The second model equation is determined based on the first formula and the second formula.

Further, the preceding tracking device of an exercise heart rate may further include a threshold pace determination module and an oxygen uptake pace determination module.

The threshold pace determination module is configured to determine the corresponding lactate threshold pace by using the standard pace of the user in the standard competition course.

The oxygen uptake pace determination module is configured to determine the maximum oxygen uptake pace of the user based on the lactate threshold pace.

Further, the preceding tracking device of an exercise heart rate may further include a heart rate ratio determination module, a standard heart rate calculation module and a standard pace calculation module.

The heart rate ratio determination module is configured to determine the target heart rate reserve ratio by using the maximum oxygen uptake of the user in the standard competition course.

The standard heart rate calculation module is configured to calculate the standard exercise heart rate of the user in the standard competition course based on the target heart rate reserve ratio and the maximum heart rate and the resting heart rate of the user.

The standard pace calculation module is configured to calculate the standard pace of the user in the standard competition course by using the standard exercise heart rate and each pre-established piecewise model equation reflecting the relationship between the user heart rate and the user pace in the standard competition course.

Further, the preceding heart rate tracking module 420 may be specifically configured to input the current real-time pace of the user into the target model equation to obtain the current initial heart rate of the user and determine the current real-time heart rate of the user according to the difference between the initial heart rate and the real-time heart rate of the user at a previous moment.

The tracking apparatus of an exercise heart rate according to this embodiment may be applied to the tracking method of an exercise heart rate according to any preceding embodiment and has corresponding functions and effects.

### Embodiment Five

FIG. 5 is a diagram illustrating the structure of an electronic device according to embodiment five of the present disclosure. As shown in FIG. 5, the electronic device includes a processor 50, a storage apparatus 51 and a communication apparatus 52. The number of processors 50 in the electronic device may be one or more. One processor 50 is used as an example in FIG. 5. The processor 50, the storage apparatus 51 and the communication apparatus 52 of the electronic device may be connected through a bus or other manners. Connecting by a bus is used as an example in FIG. 5.

As a computer-readable storage medium, the storage apparatus 51 may be configured to store a software program, a computer-executable program and a computer-executable module, for example, modules corresponding to the tracking method of an exercise heart rate in this embodiment of the present disclosure (for example, the model equation invoking module 410 and the heart rate tracking module 420 in the tracking apparatus of an exercise heart rate). The processor 50 executes various function applications and data processing of the electronic device, that is, performs the preceding tracking method of an exercise heart rate, by executing the software program, instruction and module stored in the storage apparatus 51.

The storage apparatus 51 may mainly include a program storage area and a data storage area. The program storage area may store an operating system and an application program required for implementing at least one function while the data storage area may store data created depending on the use of terminals. In addition, the storage apparatus 51 may include a high-speed random access memory and may further include a nonvolatile memory, such as at least one disk memory, flash memory or another nonvolatile solid-state memory. In some examples, the storage apparatus 51 may further include memories located remotely relative to a processor 50, and these remote memories may be connected to the electronic device via a network. Examples of the preceding network include, but are not limited to, the Internet, an intranet, a local area network, a mobile communication network and a combination thereof.

The communication apparatus 52 may be configured to perform the network connection or the mobile data connection between devices.

An electronic device according to this embodiment may be applied to execute the tracking method of an exercise heart rate according to any preceding embodiment and has corresponding functions and effects.

### Embodiment Six

Embodiment six of the present disclosure further provides a non-transitory computer-readable storage medium. The storage medium stores a computer program which, when executed by a processor, causes the processor to perform the tracking method of an exercise heart rate according to any preceding embodiment. This method includes the steps below.

If the user heart rate currently detected by the photoelectric heart rate device does not meet the preset heart rate standard, the target model equation reflecting the relationship between the user heart rate and the user pace is invoked.

The current real-time pace of the user is input into the target model equation to obtain the current real-time heart rate of the user.

Of course, in a storage medium including the computer executable instructions provided in this embodiment of the present disclosure, the computer executable instructions may execute not only the preceding method operations but also related operations in the tracking method of an exercise heart rate according to any embodiment of the present disclosure.

From the preceding description of embodiments, it will be apparent to those skilled in the art that the present disclosure may be implemented by means of software and necessary general-purpose hardware or may of course be implemented by hardware, but in many cases, the former is a preferred embodiment. Based on this understanding, the technical solutions provided by the present disclosure substantially, or the part contributing to the related art, may be embodied in the form of a software product. The software product is stored in a computer readable storage medium, such as a computer floppy disk, a read-only memory (ROM), a random access memory (RAM), a flash, a hard disk or an optical disk, and includes several instructions for enabling a computer device (which may be a personal computer, a server or a network device) to execute the method according to each embodiment of the present disclosure.

It is to be noted that units and modules included in the embodiment of the tracking apparatus of an exercise heart rate are just divided according to functional logic but are not limited to such division, as long as the corresponding functions can be implemented. In addition, the specific names of the functional units are just used for distinguishing between each other and are not intended to limit the scope of the present disclosure.

The preceding are only preferred embodiments of the present disclosure and not intended to limit the present disclosure, and for those skilled in the art, the present disclosure may have various modifications and variations. Any modifications, equivalent replacements, improvements and the like within the spirit and principle of the disclosure shall fall within the scope of protection of the disclosure.

What is claimed is:

1. A tracking method of an exercise heart rate, comprising:
in a case where a user heart rate currently detected by a photoelectric heart rate device does not meet a preset heart rate standard, invoking a pre-established target model equation reflecting a relationship between a user heart rate and a user pace; and
inputting a current real-time pace of a user into the target model equation to obtain a current real-time heart rate of the user.

2. The method according to claim 1, wherein the target model equation is a first model equation fitted according to a historical heart rate and a historical exercise speed or a second model equation determined according to a relationship between a user heart rate reserve ratio and the user heart rate and a relationship between the user heart rate reserve ratio and the user pace.

3. The method according to claim 2, wherein in a case where the target model equation is the first model equation, the target model equation is established using the following steps:
with a certain moving period and in time sequence, sequentially obtaining historical heart rates and historical exercise speeds in each continuous unit period and determining whether the historical heart rates and the historical exercise speeds in the each continuous unit period meet the following conditions:
(1) a heart rate fluctuation in the each continuous unit period is within a first preset range;
(2) a speed fluctuation in the each continuous unit period is within a second preset range; and
(3) an altitude variation in the each continuous unit period is within a third preset range;
calculating an average value of historical heart rates and an average value of historical exercise speeds in each target unit period that meets the preceding conditions and using the average value of the historical heart rates and the average value of the historical exercise speeds in the each target unit period that meets the preceding conditions as associated data points reflecting the relationship between the user heart rate and the user pace; and
using the associated data points corresponding to the each target unit period to fit the first model equation.

4. The method according to claim 2, wherein in a case where the target model equation is the second model equation, the target model equation is established using the following steps:
obtaining a first formula for calculating the user heart rate reserve ratio by using a real-time heart rate of the user, a resting heart rate of the user and a maximum heart rate of the user and a second formula for calculating the user heart rate reserve ratio by using a real-time pace of the user and a maximum oxygen uptake pace of the user; and
determining the second model equation based on the first formula and the second formula.

5. The method of claim 4, further comprising:
determining a corresponding lactate threshold pace by using a standard pace of the user in a standard competition course; and
determining a maximum oxygen uptake pace of the user based on the corresponding lactate threshold pace.

6. The method according to claim 5, before determining the corresponding lactate threshold pace by using the standard pace of the user in the standard competition course, further comprising:
determining a corresponding target heart rate reserve ratio by using a maximum oxygen uptake of the user in the standard competition course;
calculating a standard exercise heart rate of the user in the standard competition course based on the target heart rate reserve ratio and the maximum heart rate and the resting heart rate of the user; and
calculating the standard pace of the user in the standard competition course by using the standard exercise heart rate and each pre-established piecewise model equation reflecting a relationship between a user heart rate and a user pace in the standard competition course.

7. The method according to claim 1, wherein inputting the current real-time pace of the user into the target model equation to obtain the current real-time heart rate of the user comprises:
inputting the current real-time pace of the user into the target model equation to obtain a current initial heart rate of the user; and
determining the current real-time heart rate of the user according to a difference between the initial heart rate and a real-time heart rate of the user at a previous moment.

8. A tracking apparatus of an exercise heart rate, comprising:
a model equation invoking module configured to, if a user heart rate currently detected by a photoelectric heart rate device does not meet a preset heart rate standard, invoke a pre-established target model equation reflecting a relationship between a user heart rate and a user pace; and
a heart rate tracking module configured to input a current real-time pace of a user into the target model equation to obtain a current real-time heart rate of the user.

9. An electronic device, comprising:
one or more processors; and
a storage apparatus configured to store one or more programs, wherein
when executed by the one or more processors, the one or more programs cause the one or more processors to perform the tracking method of an exercise heart rate according to claim 1.

10. A non-transitory computer-readable storage medium, the storage medium storing a computer program which, when executed by a processor, causes the processor to perform the tracking method of an exercise heart rate according to claim 1.
